(19) United States
(12) Patent Application Publication
(10) Pub. No.: US 2024/0366940 A1
(43) Pub. Date: Nov. 7, 2024

(54) METHOD AND APPARATUS FOR IMPROVING KIDNEY FUNCTION

(71) Applicant: ABIOMED, Inc., Danvers, MA (US)

(72) Inventors: Jerald Curran, Danvers, MA (US); Jin Kwang Kim, Danvers, MA (US)

(73) Assignee: ABIOMED, Inc., Danvers, MA (US)

(21) Appl. No.: 18/651,905

(22) Filed: May 1, 2024

Related U.S. Application Data
(60) Provisional application No. 63/463,403, filed on May 2, 2023.

(51) Int. Cl. A61N 1/36; A61N 1/05

(52) U.S. Cl. CPC A61N 1/36053; A61N 1/0551; A61N 1/36146

(57) ABSTRACT

A method and system for improving kidney function of a patient may be provided. The method may include activating a baroreceptor, and improving a flow of blood through vasculature of the patient. This may include positioning a stent at or near a baroreceptor, the stent configured to stretch the vasculature. This may include positioning a lead at or near a baroreceptor, the lead comprising a proximal end and a distal end and an electrode connected to the distal end of the lead, wherein the electrode configured to emit one or more electrical signals, such as an intermittent electrical stimulus or a constant electrical stimulus. The stent may include a tubular body, which may be made of a flexible material.

### CROSS-REFERENCE TO RELATED APPLICATIONS

The present application claims priority to U.S. Provisional Patent Application No. 63/463,403, filed May 2, 2023, the contents of which are incorporated by reference herein in its entirety.

### TECHNICAL FIELD

Embodiments disclosed herein relate to medical devices, such as stents, and devices and methods for improving kidney function.

### BACKGROUND

Stents may be introduced into a patient's vasculature to reinforce, support, repair and/or enhance performance. For example, stents may be used within the patient's vasculature to keep a vessel from narrowing or closing. In such an example, once in place, the stent can reinforce that part of the vessel while still allowing blood to flow through. Stents may be placed in various places within the patient's vasculature. Such stents may be placed by positioning the stent at the desired location and using a balloon catheter to expand the stent. Other stents may be self-expanding. The stent may remain in the expanded state while inside of the patient.

Baroreceptors derive information from blood pressure and convey such information to the autonomic nervous system.

### BRIEF SUMMARY

In various aspects, a method for improving kidney function may be provided. The method may include activating a baroreceptor. The method may include improving a flow of blood through vasculature of the patient.

Activating the baroreceptor may include positioning a stent at or near a baroreceptor. The stent may be configured to stretch the vasculature. The stent may be positioned at or near a superior vena cava of the patient.

The method may include positioning a lead at or near a baroreceptor. The lead may include a proximal end and a distal end. The lead may include an electrode connected to the distal end of the lead. The electrode may be configured to emit one or more electrical signals. The electrode may be configured to emit an intermittent electrical stimulus.

Activating the baroreceptor may include positioning a lead at or near a baroreceptor. The lead may include a proximal end and a distal end. The lead may include an electrode connected to the distal end of the lead. The electrode may be configured to emit one or more electrical signals. The electrode may be configured to emit an intermittent electrical stimulus.

In various aspects, a system may be provided. The system may include a stent. The stent may include a tubular body, which may include a flexible material. The stent may be configured to be positioned within a patient's vasculature at or near a baroreceptor. The system may include a lead. The lead may include a proximal end and a distal end. The lead may include an electrode connected to the distal end of the lead. The electrode may be configured to emit one or more electrical signals to vasculature surrounding the electrode. The stent may be configured to stretch the vasculature. The lead may be configured to electrically stimulate the baroreceptor.

The stent may be positioned at or near a superior vena cava. The lead may be positioned at or near a superior vena cava.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIGS. 1A and 1B are illustrations showing a patient's heart with a stent inserted into a superior vena cava (1A) or aortic arch (1B);

FIG. 2A shows an illustrative side view of a stent being placed into a patient's vasculature;

FIG. 2B shows an illustrative side view of a stent being placed into a patient's vasculature;

FIG. 2C shows an illustrative side view of a stent in a patient's vasculature;

FIG. 2D shows an illustrative side view of a stent in a sheath;

FIG. 2E shows an illustrative side view of a stent exiting a sheath;

FIG. 3 shows a patient's heart with a lead inserted into a superior vena cava;

FIG. 4 shows a patient's heart with a stent and a lead inserted into a superior vena cava; and

FIG. 5 illustrates a method according to one embodiment.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

As it is known, baroreceptors derive information from blood pressure and convey such information to the autonomic nervous system. The autonomic nervous system, in turn, may adjust total peripheral resistance and cardiac output to maintain blood pressure within a normalized range. As is known, there are two types of baroreceptors in the heart. High-pressure arterial baroreceptors are located within the carotid sinuses and the aortic arch. Low-pressure volume receptors are located within the atria, ventricles, and pulmonary vasculature. Low-pressure volume receptors inform the autonomic nervous system of blood volume within the system. Specifically, a low-pressure volume baroreceptor is located in the top of the right atrium near the connection to the superior vena cava and maybe even in the superior vena cava (SVC).

In some instances, stretching a patient's vasculature may assist with activating the baroreceptors in the heart. For example, both types of baroreceptors, high-pressure arterial baroreceptors and low-pressure volume receptors, may be stimulated by the stretching of the vessel wall. Both types of baroreceptors also may be activated via an electrical stimulus.

In some instances, by activating the low-pressure volume baroreceptor, a decrease in sympathetic activation and the reflow of blood to the kidneys may be improved. Accordingly, the inventors have recognized the benefit of stretching the vessel (while simultaneously allowing blood to flow therethrough) in order to activate low-pressure volume baroreceptor(s). For example, as disclosed herein, a stent may be placed near the superior vena cava to activate the low-pressure volume baroreceptor by stretching the vessel. The inventors have also recognized that activating the low-pressure volume baroreceptor via an electrical lead may result in improved kidney functionality. In some embodiments, stent placement may be used in combination with one or more electrode leads to activate the low-pressure baroreceptors.

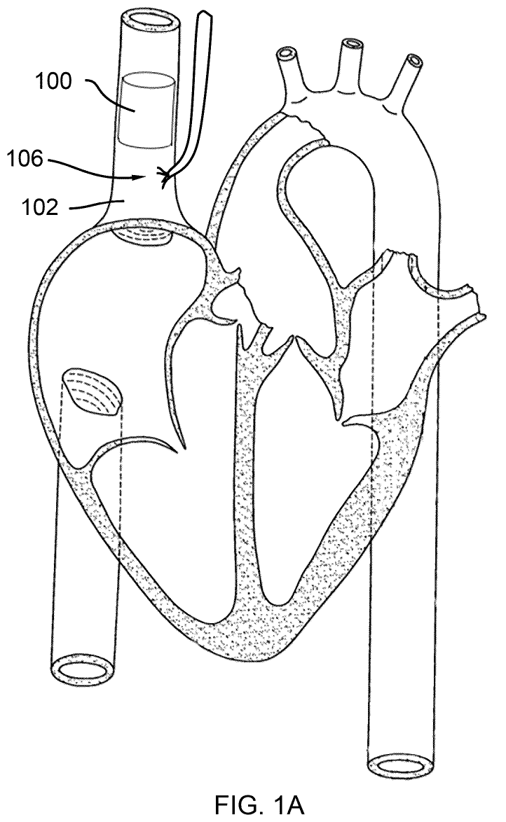

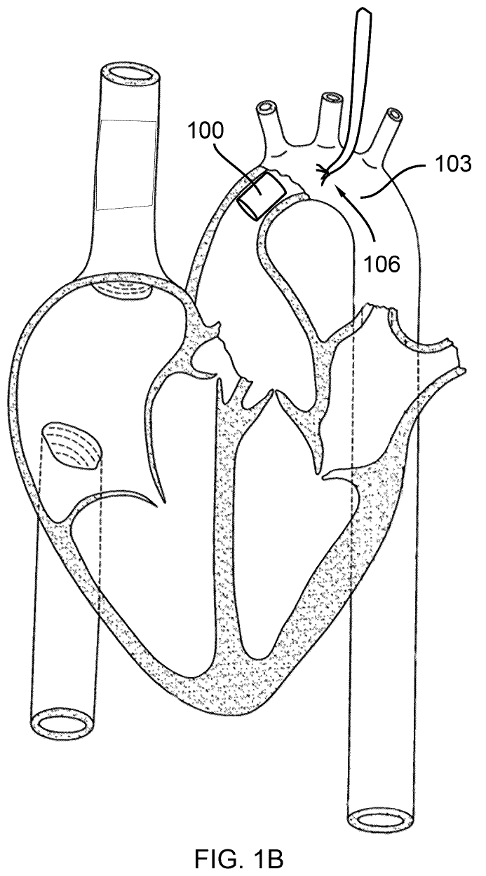

Turning to the figures, FIG. 1A shows an illustrative stent (100) in a patient according to one embodiment of the present disclosure. As will be appreciated, the stent (100) may include any suitable configuration. For example, the stent may be tubular shaped device (e.g., the stent may have a tubular body). The stent may be expandable. The stent may be formed of an expandable material. The stent may be formed of a metal mesh. As will also be appreciated, the stent (100) may be formed of a plurality of struts, the struts having any suitable strut geometry. For example, the stent (100) may have Z-shaped struts. The stent (100) may have a closed cell wire strut structure. Alternatively, the stent (100) may have braided struts.

As shown in FIG. 1A, in some embodiments, the stent (100) may be positioned in the patient's vasculature to activate a baroreceptor. For example, as shown in FIG. 1A, the stent (100) may be positioned in the superior vena cava (102) to stretch the vessel. In such an example, by stretching the superior vena cava (102), the baroreceptor located nearby may be activated. By activating this baroreceptor with the stent (100), blood may still flow through the SVC (102) (e.g., threw the stent) while sympathetic activation is decreased. Thus, blood reflow to the kidneys may be improved.

Although the stent is shown as being positioned in the SVC in FIG. 1A, it will be appreciated that the stent (100) may be positioned in other suitable portions of the patient's vasculature to activate baroreceptors (106). For example, in another embodiment, the stent may be placed on the left side of the heart (e.g., in or near the aortic arch (103) (see FIG. 1B) in order to activate left-sided baroreceptors.

In some embodiments, the material of the stent (100) may be flexible enough to modulate (e.g., expand from a delivery configuration to a deployed configuration) while maintaining the position of the stent and stretching the patient's vasculature. As will also be appreciated, the stent (100) may be made of other suitable materials. In some embodiments, the entire stent may be formed of the same material, although the stent may be formed of more than one material. The stent also may be formed of the same material but have different material properties thereout. In one embodiment, the struts of the stent (100) may be made of a material that is different than the material of the ends of the stent (100). In another embodiment, the ends of the stent (100) may be made of a stiffer material to maintain positioning of the stent while blood is flowing through the vessel. As will be appreciated, the stent (100) may also have any suitable radial stiffness and/or may have sections with different radial stiffnesses.

In some embodiments, the stents may be bare-metal stents or drug-eluting stents. In some embodiments, the stent (100) may be a bare-metal stent for short-term use. In some embodiments, the drug-eluting stents may have a coating that slows down the process of the vasculature re-narrowing. Thus, in some embodiments, the stent (100) may be drug-eluting for long-term usage. For example, the stent (100) may be drug-eluting for usage longer than six months.

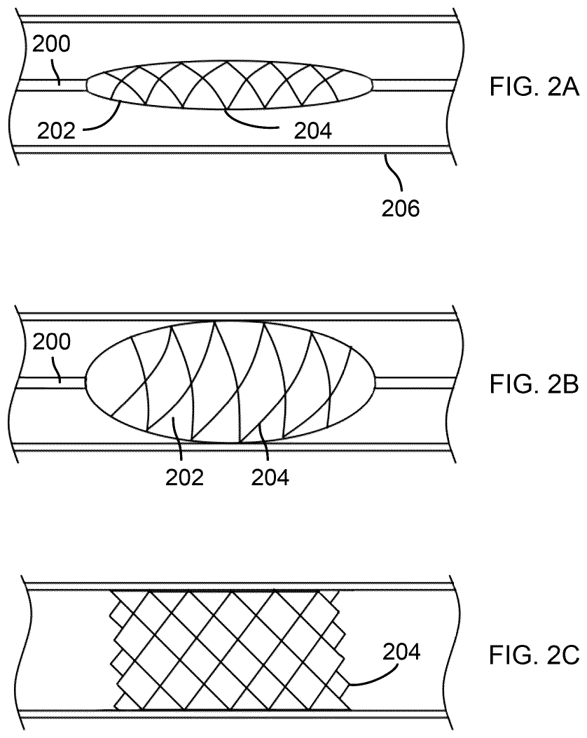

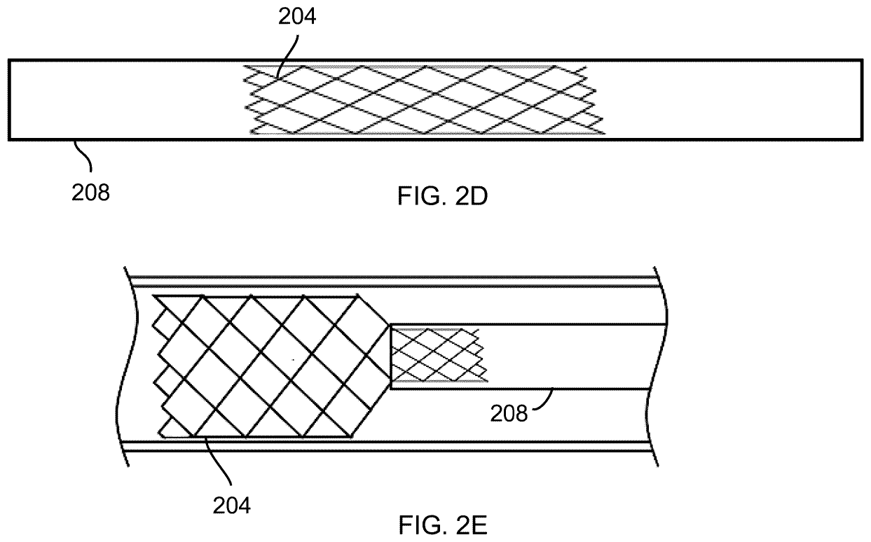

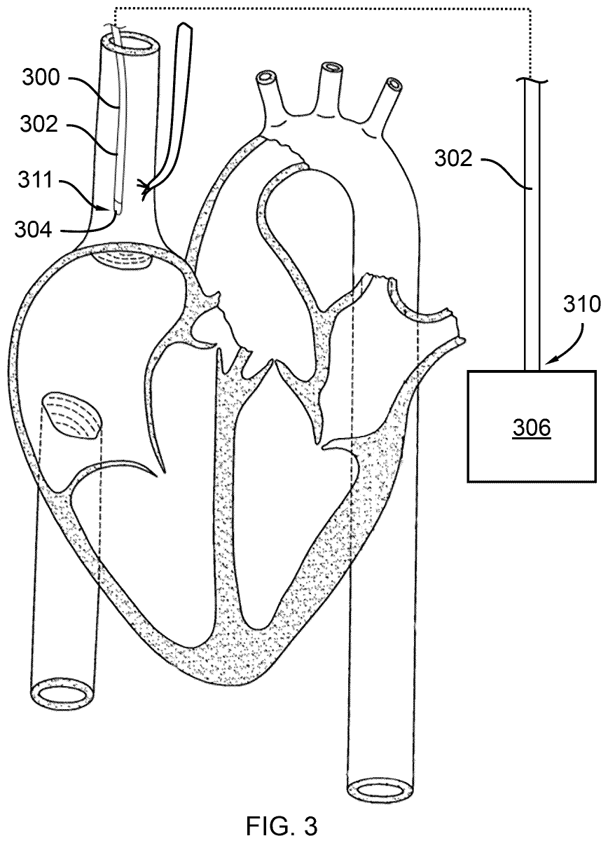

In some embodiments, as illustrated in FIG. 2A-FIG. 2C, the stent (100) may be inserted using angioplasty. For example, during such a procedure, a catheter (200) may be inserted into the patient's vasculature (206) and guided to the desired location where the stent will be delivered. In some instances, the catheter may be inserted via the femoral artery. Alternatively, to access the superior vena cava, the catheter also may be inserted via the interior jugular vein. As shown in FIG. 2A, the catheter (200) may be advanced through the patient's vasculature in a delivery configuration to the desired location of the stent (e.g., the SVC). As will be appreciated, in the delivery configuration, the stent may be placed around a catheter, such as a balloon catheter, and may have a diameter that is larger than that of the stent in the completely collapsed state. FIG. 2B shows an inflatable balloon (202) of the catheter (200) positioning inside the stent, with the balloon (202) in an inflated state. As will be appreciated, the inflatable balloon (202) may be positioned along at least a portion of the length of the catheter (200). As shown in FIG. 2B, the balloon (202) may be inflated to expand the stent (100) to an expanded, deployed configuration. FIG. 2C shows the stent in the deployed configuration, after the balloon (202) has been deflated and the catheter (200) has been removed from the patient's vasculature (206), leaving the stent (100) at the desired location (e.g., the SVC).

In some embodiments, the catheter (200) and the balloon (202) may be inserted first. The balloon (202) is the inserted and the stent (100) is placed once the balloon (202) is inflated and expanding the vasculature (206).

As will be appreciated, the stent may be delivered in other manners. For example, in some embodiments, the stent may be introduced into the catheter and “hatched” out of the catheter and into the desired location (e.g., the SVC). As shown in FIG. 2D, for example, in other embodiments, the stent (204) may be compressed into a delivery configuration by inserting the stent into a sheath (208). As will be appreciated, the length of the sheath (208) may be any suitable length for insertion into a patient's vasculature. As shown in FIG. 2E, the sheath (208) then may be inserted into the patient and the stent (204) deployed (e.g., hatched) from the sheath (208) once the sheath (208) is at the desired location. In some embodiments, the stent (204) may expand as the sheath (208) is pulled back. In other embodiments, the stent (204) may be pushed out of the sheath (208). The stent (204) may be expanded into the deployed configuration. The sheath (208) may then be removed from the patient, leaving the stent in the desired location. As will be appreciated, any suitable method of delivering the stent (100) may be used.

According to another aspect, the baroreceptors may be stimulated via electrical stimulus. As shown in FIG. 3, a lead (300) may be positioned in a patient's vasculature. The lead (300) may have a proximal end (310) and a distal end (311). The lead (300) may include a cable (302) and an electrode (304) at the distal tip (e.g., at distal end (311)) of the cable (302). In one embodiment, only one cable (302) and/or one electrode (304) may be positioned in the patient. In other embodiments, there may be more than one electrode (304) on the lead (300). In other embodiments, there may be more than one lead (300) positioned in the same vasculature. As shown in FIG. 3, in some embodiments, the cable (302) may be connected to an external controller (306). For example, in FIG. 3, the proximal end (310) of the lead may be operably coupled to a controller (306). The controller (306) may control the electrical signals that are emitted from the electrode (304).

As seen in FIG. 3, the electrode (304) may be positioned in the superior vena cava. In some embodiments, the electrode may send one or more electrical signals to electrically stimulate the vasculature near the electrode (e.g., surrounding the electrode), and thereby stimulate one or more baroceptors. For example, the electrical signals may stimulate baroreceptors that are near the superior vena cava. In some embodiments, the electrical signals may be a constant stimulus. In other embodiments, the electrical signals may be emitted intermittently. For example, the controller (306) may cause the electrode (304) to emit electrical signals in response to decreased blood flow through the superior vena cava. In embodiments where there is more than one lead (300), the leads (300) may be positioned such that the electrodes (304) are stimulating different parts of the vasculature.

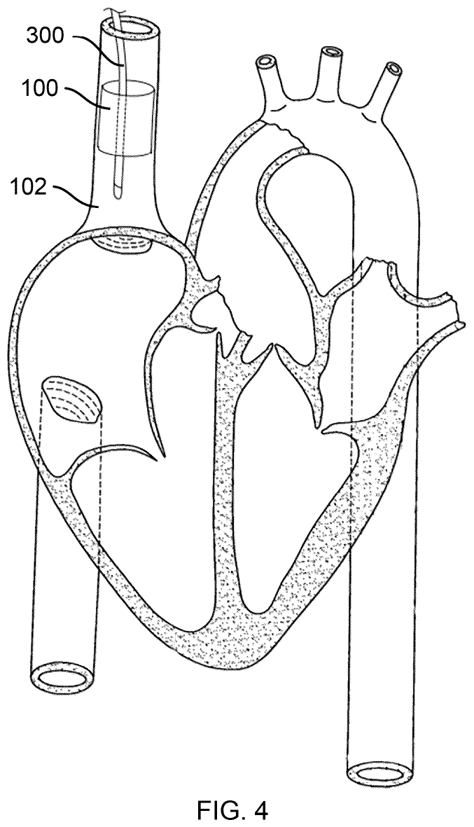

In some embodiments, baroreceptors may be stimulated only via stretching (e.g., via the stent) or via only electrical stimulation (e.g., via one or more leads). In other embodiments, the stent (100) and the leads (300) may be used in conjunction with each other. For example, in some embodiments, both the stent (100) and the lead (300) may be positioned in the vessel at the same time for stimulating the baroreceptors. For example, both the stent (100) and the lead (300) may be positioned at or near the superior vena cava (102). In some embodiments, the lead (300) may pass through the stent (100), as shown in the embodiment in FIG. 4. In all of these aforementioned embodiments, one or more stents (100) may be used and/or one or more leads (300) may be used for baroreceptor stimulation.

Additionally, in some embodiments, the lead (300) may be positioned outside of the superior vena cava (102). For example, a small incision may be made in the patient's skin and the lead (300) may be inserted into the incision. The electrode (304) of the lead (300) may be positioned such that it is at or near the superior vena cava (102). As will be appreciated, in some embodiments, the lead (300) may be positioned in any suitable position in order to stimulate the superior vena cava. Additionally, in some embodiments, more than one lead (300) may be inserted into the incision. In some embodiments, there may be more than one incision.

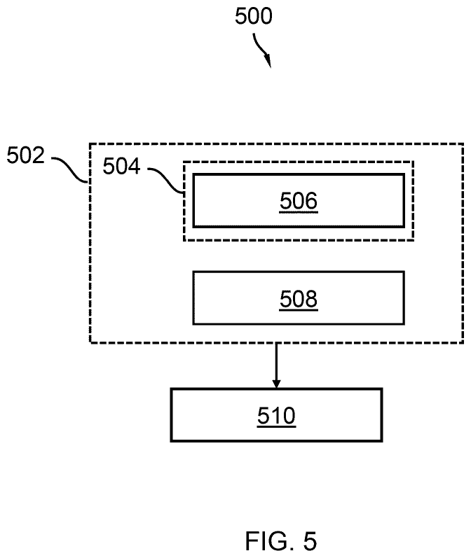

As shown in FIG. 5, the method (500) of improving kidney function according to some embodiments is disclosed. In some embodiments, the method may include activating (502) the baroreceptor. In some embodiments, the step of activating the baroreceptor may include positioning (504) a stent at or near a baroreceptor. In such embodiments, the stent may be configured to stretch the vasculature. In some embodiments, positioning the stent at or near a baroreceptor may include positioning (506) the stent at or near the superior vena cava. In other embodiments, the step of activating the baroreceptor may include positioning (508) a lead at or near a baroreceptor. The lead may include an electrode at the distal end. The electrode may emit one or more electrical signals. In some embodiments, the step of activating the baroreceptor(s) may include positioning the stent at or near the superior vena cava and positioning a lead at or near a baroreceptor. In some embodiments, the method may also include the step of improving (510) a flow of blood through the vasculature.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method for improving kidney function of a patient, comprising;
activating a baroreceptor; and
improving a flow of blood through vasculature of the patient.

2. The method of claim 1, wherein activating the baroreceptor includes positioning a stent at or near a baroreceptor, the stent configured to stretch the vasculature.

3. The method of claim 2, wherein the stent is positioned at or near a superior vena cava of the patient.

4. The method of claim 2, further comprising positioning a lead at or near a baroreceptor, the lead comprising a proximal end and a distal end and an electrode connected to the distal end of the lead, wherein the electrode configured to emit one or more electrical signals.

5. The method of claim 4, wherein the electrode is configured to emit an intermittent electrical stimulus.

6. The method of claim 1, wherein activating the baroreceptor includes positioning a lead at or near a baroreceptor, the lead comprising a proximal end and a distal end and an electrode connected to the distal end of the lead, wherein the electrode is configured to emit one or more electrical signals.

7. The method of claim 6, wherein the electrode is configured to emit a constant electrical stimulus.

8. A system comprising:
a stent comprising a tubular body made of a flexible material and configured to be positioned within a patient's vasculature at or near a baroreceptor; and
a lead having a proximal end and a distal end, an electrode connected to the distal end of the lead, the electrode configured to emit one or more electrical signals to vasculature surrounding the electrode;
wherein the stent is configured to stretch the vasculature and the lead is configured to electrically stimulate the baroreceptor.

9. The system of claim 8, wherein the stent is positioned at or near a superior vena cava.

10. The system of claim 8, wherein the lead is positioned at or near a superior vena cava.
